(19) United States
(12) Patent Application Publication
(10) Pub. No.: US 2016/0190739 A1
(43) Pub. Date: Jun. 30, 2016

(54) MEDICAL MODULE CONNECTION BASE AND MEDICAL MODULE EXPANSION SYSTEM

(71) Applicant: SHENZHEN MINDRAY BIO-MEDICAL ELECTRONICS CO., LTD., Shenzhen (CN)

(72) Inventors: Jianfeng Jiang, Shenzhen (CN); Lei Qing, Shenzhen (CN)

(21) Appl. No.: 14/885,906

(22) Filed: Oct. 16, 2015

Related U.S. Application Data
(63) Continuation of application No. PCT/CN2013/083101, filed on Sep. 9, 2013.

(30) Foreign Application Priority Data
Apr. 16, 2013 (CN) 201310132057.2

(51) Int. Cl. H01R 13/631; H01R 13/46; A61B 17/00

(52) U.S. Cl. CPC H01R 13/631; A61B 17/00; H01R 13/46

(57) ABSTRACT

Disclosed is a medical module connection base (20,30), comprising a support wall (22,31) for supporting a medical module (10), a side wall (23,32) arranged on the support wall (22,31), and a signal transmission assembly which is arranged on the support wall (22,31) or the side wall (23,32) and communicates signals with the medical module (10). Further disclosed is a medical module expansion system, comprising a first medical module connection base (30) and a second medical module connection base (20) which are connected to and combined with a medical module (10), so that the medical module (10) can be functionally expanded and exchange data with external equipment conveniently.

### FIELD OF THE INVENTION

The present disclosure relates to a medical module connection base and a medical module expansion system.

### BACKGROUND OF THE INVENTION

An existing medical module, such as a monitor module, is not provided with a variety of extended functions. With respect to the switch between bedside monitoring and patient transfer monitoring, it is a tedious work due to mounting or dismounting stationary structures or plugging peripheral cables.

### SUMMARY OF THE INVENTION

According to an embodiment of the present disclosure, a medical module connection base is provided, including a support wall for supporting a medical module, a side wall arranged on the support wall, and a signal transmission assembly which is arranged on the support wall or the side wall and communicates signals with the medical module.

In a further improvement of the medical module connection base, an upside of the support wall may be provided with a second guide structure cooperated with a first guide structure of the medical module.

In a further improvement of the medical module connection base, the signal transmission assembly may include at least one input and/or output interface and a first socket, and the first socket is connected to the medical module and electrically connected to the input and/or output interface.

In a further improvement of the medical module connection base, the first socket may include a plug-in interface.

In a further improvement of the medical module connection base, the input and/or output interface may further include at least one of an alternating current input socket, a VGA interface, a multifunction interface, a USB interface and a network interface.

In a further improvement of the medical module connection base, the signal transmission assembly may be a plug-in interface.

In a further improvement of the medical module connection base, a downside of the support wall may be provided with a third guide structure.

In a further improvement of the medical module connection base, the medical module connection base may further include a handle arranged on the support wall or the side wall.

In a further improvement of the medical module connection base, the medical module connection base may further include a rear side wall which is arranged on the support wall and provided with a hook.

According to an embodiment of the present disclosure, a medical module expansion system is provided, including a first medical module connection base and a second medical module connection base. The first medical module connection base may include a first support wall, a first side wall arranged on the first support wall, a first signal transmission assembly arranged on the first side wall, and a second guide structure arranged on an upside of the first support wall. The second medical module connection base may include a second support wall, a second side wall arranged on the second support wall, a second signal transmission assembly arranged on the second side wall, and a fourth guide structure and a third guide structure arranged respectively on an upside and a downside of the second support wall. The second guide structure may match the third guide structure, the fourth guide structure may match the first guide structure of a medical module, and the first signal transmission assembly may communicate signals with the medical module via the second signal transmission assembly.

In a further improvement of the medical module expansion system, the second signal transmission assembly may be a second plug-in interface, the first signal transmission assembly may comprise at least one input and/or output interface and a first plug-in interface connected with the second plug-in interface, and the first plug-in interface may be electrically connected with the input and/or output interface.

According to the connection base and the medical module expansion system of the present disclosure, by means of connecting and combining the medical module and the medical module connection base having a signal transmission assembly, the medical module could communicate data with peripherals, resulting in convenience of extending functions of the medical module, and flexibility in providing a plurality of clinical applications and function combination. In addition, the operation of plugging peripheral cables may also be simplified when monitoring beside hospital beds or patient transfer, thus making the operation convenient and efficient, shortening the operation time and improving the transfer efficiency.

### BRIEF DESCRIPTION OF THE DRAWINGS

FIG. 1 is a schematic perspective view of combination of a first medical module connection base and a medical module according to a first embodiment of the present disclosure.

FIG. 2 is a schematic exploded view of the first medical module connection base and the medical module shown in FIG. 1.

FIG. 3 is a schematic perspective view of combination of a second medical module connection base and a medical module according to a second embodiment of the present disclosure.

FIG. 4 is a schematic exploded view of the second medical module connection base and the medical module shown in FIG. 3.

FIG. 5 is a schematic exploded view of the rear of the combination shown FIG. 3.

FIG. 6 is a schematic view of FIG. 3 seen from a tilted angle.

FIG. 7 is a schematic perspective view of combination of a medical module expansion system and a medical module according to a third embodiment of the present disclosure.

FIG. 8 is a schematic view of the rear of the combination shown in Fig.

### DETAILED DESCRIPTION OF THE INVENTION

### First Embodiment

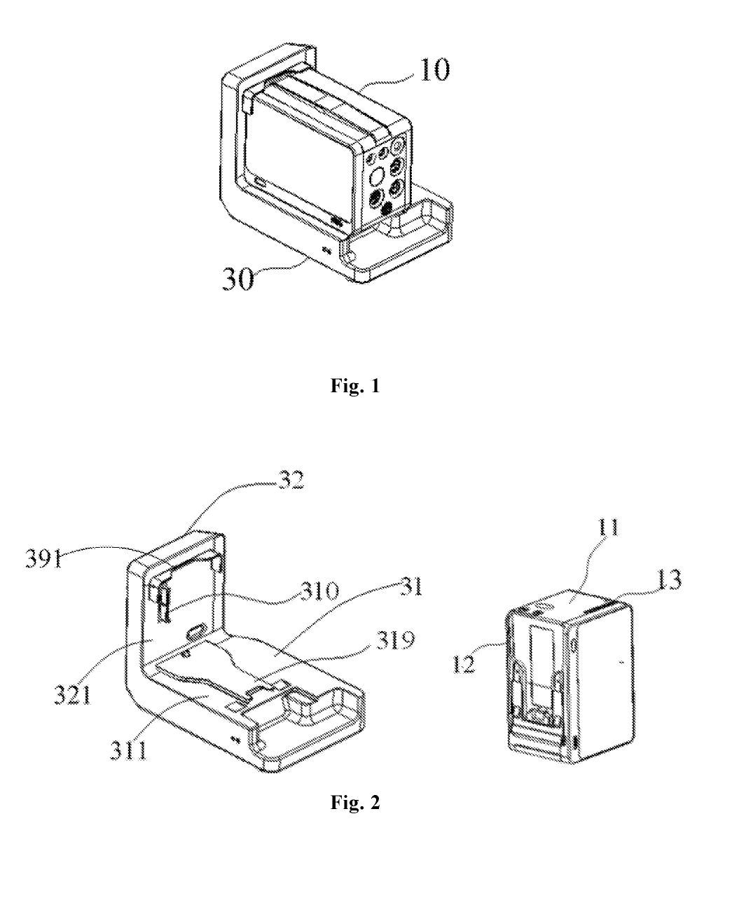

Referring to FIG. 1 and FIG. 2, a first medical module connection base 30 may be combined with a medical module 10 according to the first embodiment.

The medical module 10 may have a housing 11 which is provided with a first guide slot 12 at the bottom surface and a data interface 13 at a side face. In the embodiment, the medical module 10 is a monitor module, and the data interface 13 is a plug-in interface female socket.

The first medical module connection base 30 may include a first support wall 31 and a first side wall 32 which is vertical to the first support wall 31. The first support wall 31 may be provided with a first guide rail 319 protruded on the inner bottom surface 311. The first side wall 32 may be provided with a first socket 310 and a guide rib 391 at the inner side face 321. In the embodiment, the first socket 310 could be a plug-in interface male socket.

Please referring to FIG. 8, the first medical module connection base 30 may be provided with input and/or output interfaces including an alternating current input socket 33, a VGA interface 34, a multifunction interface 35, a USB interface 36 and a RJ45 network interface 37 at the rear side thereof. The VGA interface 34 may be used for connecting with an external display. The multifunction interface 35 may be used for connecting with other monitors. The first socket 310 may be electrically connected to the input and/or output interfaces. The medical module 10 and the first medical module connection base 30 may be electrically connected through the first socket 310, so that the data from the medical module 10 may be selectively exported to each input and/or output interface. In the embodiment, the first socket 310 together with the input and/or output interfaces may form a signal transmission assembly of the first medical module connection base 30. Of course, the first socket 310 and the input and/or output interfaces may be disposed at any position of the first support wall 31 or the first side wall 32 as required.

When connecting the first medical module connection base 30 with the medical module 10, the medical module 10 could be slipped into or slipped off the first medical module connection base 30 via the guiding of the first guide slot 12 and the first guide rail 319. The first socket 310 can be connected with the data interface 13 in a pluggable manner, so that the medical module 10 and the first medical module connection base 30 could be combined into a hospital bedside monitor which can be fixedly disposed beside the bed through the first medical module connection base 30 and provides other extended functions for the monitor module and prepares for next transfer during independent monitoring.

### Second Embodiment

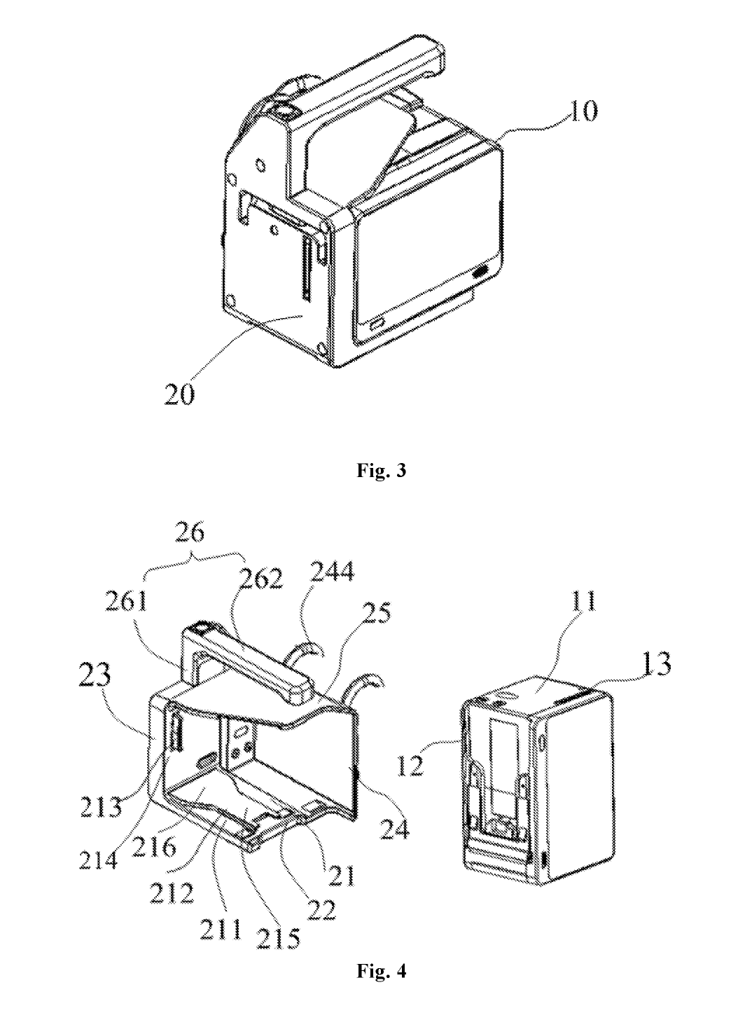

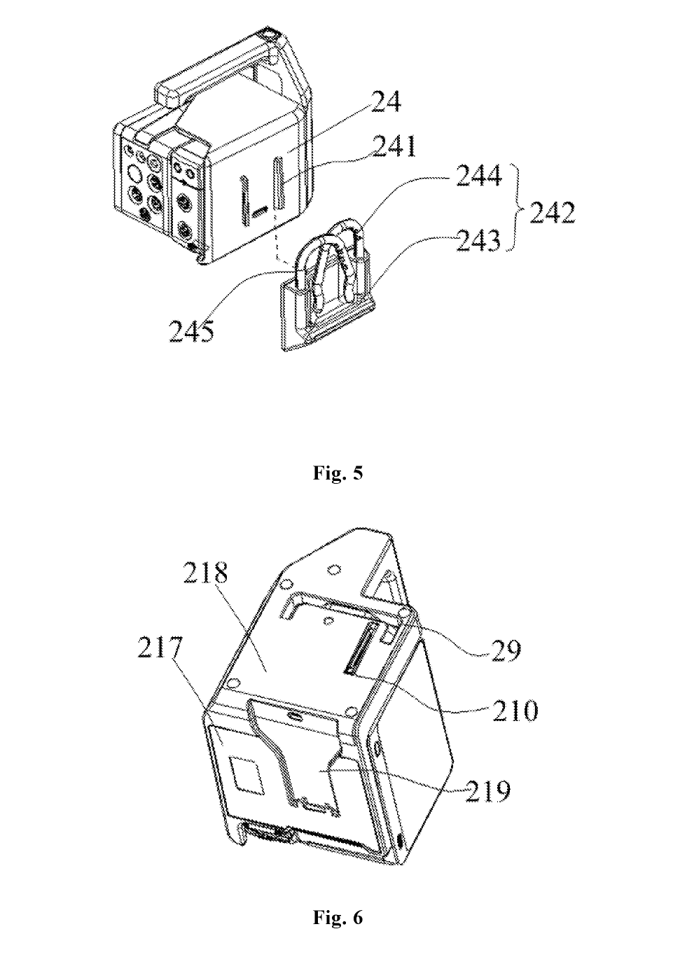

Referring to FIG. 3 to FIG. 5, a second medical module connection base 20 may be combined with the medical module 10 according to the second embodiment.

The second medical module connection base 20 may include a holding space 21 and a handle 26 disposed above the holding space 21. The holding space 21 may be a semi-enclosed space formed by a second support wall 22, a second side wall 23, a rear side wall 24 and an incomplete upper wall 25. The holding space 21 has an inner bottom surface 211 and an inner side face 214. A second guide rail 212 may be protruded on the inner bottom surface 211. The inner side face 214 may be provided with a second plug-in interface male socket 213. In the embodiment, the second plug-in interface male socket 213 could be a plug-in interface male socket. The second guide rail 212 may include a narrow part 215 near the outer side and a wide part 216 near the inner side. The narrow part 215 may be gradually widened towards the wide part 216. The second guide rail 212 may be adapted to the first guide slot 12 of the medical module 10. Of course, the second plug-in interface male socket 213 may be disposed at any position of the second support wall 22, the second side wall 23, the rear side wall 24 or the upper wall 25 as required.

The handle 26 of the second medical module connection base 20 may include a vertical part 261 connected with an end part of the second side wall 23 and a horizontal part 262 connected with vertical part 261, which facilitates carrying the medical module system by hand. Of course, the handle 26 may also be disposed on the second support wall 22, the rear side face 24 or the upper wall 25 as required.

The medical module 10 may be slipped into or slipped off the holding space 21 of the second medical module connection base 20 via the guiding of the first guide slot 12 and the second guide rail 212. The second plug-in interface male socket 213 could be connected with the data interface 13 in a pluggable manner, so that the medical module 10 and the second medical module connection base 20 could be mounted to be a transfer monitoring device having a handle which could be mounted to a cross bar of the bed or a bed tail plate through the handle during the process of transfer. Furthermore, as shown in FIG. 8, the second medical module connection base 20 could be plugged with other parameter modules 40 to extend functions of the medical module 10. With electrical connection obtained by plugging the second plug-in interface male socket 213 into the data interface 13, the extended parameter modules 40 could transfer data to the medical module 10.

The second medical module connection base 20 may further include two straight rails 241 protruded on the back side of the rear side wall 24 and a hook structure 242. The hook structure 242 may include a support plate 243 and two hooks 244 fixedly arranged on the support plate 243. The support plate 243 is provided with two grooves 245 at the bottom surface. One end of the groove may be straightly connected to the bottom end of the support plate 243, and the other end is enclosed. With the cooperation of the grooves 245 and the straight rails 241, the hook structure 242 may be detachably connected to the side wall 24. With the two hooks 244, the combination of the second medical module connection base 20 and the medical module 10 in the embodiment could be mounted at the cross bar of the bed or the bed tail plate (not shown in figures) during the process of transfer.

Referring to FIG. 6, a second guide slot 219 is arranged on an outer bottom surface 217 of the second support wall 22, a second plug-in interface female socket 210 and a guiding groove 29 is arranged on an outer side face 218 of the second side wall 23, thereby achieving combination of the second medical module connection base 20 and the first medical module connection base 30. In the embodiment, the second plug-in interface female socket 210 and the second plug-in interface male socket 213 may form a second signal transmission assembly.

### Third Embodiment

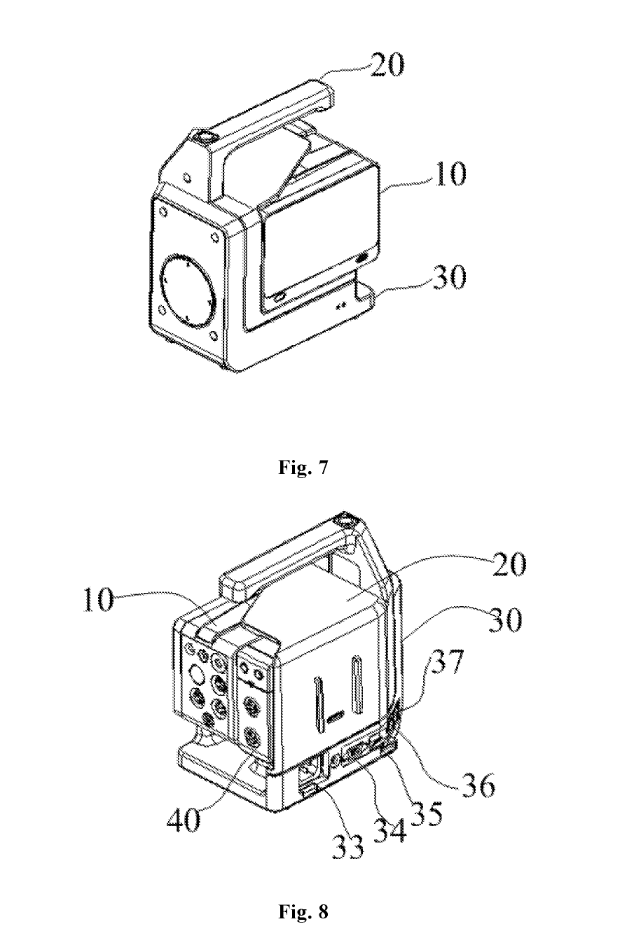

Referring to FIG. 7 and FIG. 8, a medical module expansion system having the first medical module connection base 30 and the second medical module connection base 20 is provided according to the third embodiment.

Through the guiding of the second slot 219 and the first guide rail 319 of the first medical module connection base 30, the second medical module connection base 20 could be slipped into the first medical module connection base 30. The guiding groove 29 may match a guide rib 391, and the second plug-in interface female socket 210 may, with the guide slot, be electrically connected to the first socket 310. With the cooperation of the first guide slot 12 of the medical module 10 and the second guide rail 212 of the second medical module connection base 20, the medical module 10 may be slipped into the second medical module connection base 20, and the second plug-in interface male socket 213 may be plugged into the data interface 13 of the medical module 10, forming electrical connection. Accordingly, the medical module 10 could be electrically connected to the first medical module connection base 30, so that the data of the medical module 10 could be exported to each input and/or output interface for interaction. In such way, formed by the medical module 10, the second medical module connection base 20 and the first medical module connection base 30, a portable hospital bedside monitor could be fixedly disposed beside the bed through the first medical module connection base 30 and provides other extended functions to the monitor module 10. It could also be used for preparing next transfer during independent monitoring.

In conclusion, with the guiding of the guide structure and the pluggable electrical connection between the data interface 13 and the signal transmission assembly, the medical module may be easy to be detachably combining with the connection base, so that the signals of the medical module could be exported through the connection base, and the medical module and the connection base could form a medical module system with extended functions.

The foregoing descriptions of specific examples are intended to illustrate, appreciate and not to limit the present disclosure. Various changes and modifications may be made to the aforesaid embodiments by those skilled in the art without departing from the spirit of the present disclosure.

What is claimed is:

1. A medical module connection base, comprising a support wall for supporting a medical module, a side wall arranged on the support wall, and a signal transmission assembly which is arranged on the support wall or the side wall and communicates signals with the medical module.

2. The medical module connection base according to claim 1, wherein an upside of the support wall is provided with a second guide structure cooperated with a first guide structure of the medical module.

3. The medical module connection base according to claim 2, wherein the signal transmission assembly comprises at least one input and/or output interface and a first socket, and the first socket is connected to the medical module and electrically connected to the input and/or output interface.

4. The medical module connection base according to claim 3, wherein the first socket comprises a plug-in interface.

5. The medical module connection base according to claim 4, wherein the input and/or output interface comprises at least one of an alternating current input socket, a VGA interface, a multifunction interface, a USB interface and a network interface.

6. The medical module connection base according to claim 2, wherein the signal transmission assembly comprises a plug-in interface.

7. The medical module connection base according to claim 6, wherein a downside of the support wall is provided with a third guide structure.

8. The medical module connection base according to claim 1, further comprising a handle arranged on the support wall or the side wall.

9. The medical module connection base according to claim 1, further comprising a rear side wall which is arranged on the support wall and provided with a hook.

10. A medical module expansion system, comprising a first medical module connection base and a second medical module connection base;
the first medical module connection base comprising a first support wall, a first side wall arranged on the first support wall, a first signal transmission assembly arranged on the first side wall, and a second guide structure arranged on an upside of the first support wall;
the second medical module connection base comprising a second support wall, a second side wall arranged on the second support wall, a second signal transmission assembly arranged on the second side wall, and a fourth guide structure and a third guide structure arranged respectively on an upside and a downside of the second support wall; and
the second guide structure matching the third guide structure, the fourth guide structure matching the first guide structure of a medical module, and the first signal transmission assembly communicating signals with the medical module via the second signal transmission assembly.

11. The medical module expansion system according to claim 10, wherein the second signal transmission assembly is a second plug-in interface, the first signal transmission assembly comprises at least one input and/or output interface and a first plug-in interface connected with the second plug-in interface, and the first plug-in interface is electrically connected with the input and/or output interface.
